(19) United States
(12) Patent Application Publication
(10) Pub. No.: US 2011/0195885 A1
(43) Pub. Date: Aug. 11, 2011

(54) Vegetable oil-based hydraulic fluid and transmission fluid

(75) Inventor: Otto Botz, Root (CH)

(73) Assignee: venyard GmbH

(21) Appl. No.: 13/066,320

(22) Filed: Apr. 12, 2011

Related U.S. Application Data
(63) Continuation-in-part of application No. PCT/EP2009/007341, filed on Oct. 13, 2009.

(30) Foreign Application Priority Data
Oct. 14, 2008 (CH) 1714/08

(51) Int. Cl. C10M 105/26; C07C 57/02

(52) U.S. Cl. 508/506; 554/224

(57) ABSTRACT

The invention relates to using vegetable oil having a natural viscosity index (VI) of greater or equal to 200 as a pressure medium in hydraulic systems and as a transmission fluid. The vegetable oil has a portion of monounsaturated fatty acids of at least 80%, a portion of double unsaturated fatty acids of 1-10% at maximum, and a portion of triple unsaturated fatty acids of less than 1%, preferably less than 0.1%. Part of the vegetable oil can be used in the form of an unsaturated ester of the vegetable oil. The vegetable oil can also contain an additive in a portion of 2-5% by weight selected from anti-oxidants, copper deactivators, anti-corrosion agents, wear protection agents and anti-foaming agents. The shear stability of the vegetable oil used according to the invention equals 0.7% or less, measured over 20 hours of use.

### CROSS REFERENCE TO RELATED APPLICATION

This application is filed under 35 U.S.C. §111(a) and is based on and hereby claims priority under 35 U.S.C. §120 and §365(c) from International Application No. PCT/EP2009/007341, filed on Oct. 13, 2009, and published as WO 2010/043371 A1 on Apr. 22, 2010, which in turn claims priority from Swiss Application No. 1714/08, filed on Oct. 14, 2008. This application is a continuation-in-part of International Application No. PCT/EP2009/007341, which is a continuation of Swiss Application No. 1714/08. International Application No. PCT/EP2009/007341 is pending as of the filing date of this application, and the United States is an elected state in International Application No. PCT/EP2009/007341. This application claims the benefit under 35 U.S.C. §119 from Swiss Application No. 1714/08, filed on Oct. 14, 2008, in Switzerland. The disclosure of each of the foregoing documents is incorporated herein by reference.

### TECHNICAL FIELD

The present invention relates to the use of a vegetable oil of a specific composition as a hydraulic fluid and transmission fluid.

### BACKGROUND

A hydraulic fluid is generally understood to be a fluid that is needed for transmitting energy in hydraulic systems. Hydraulic fluids must meet a large number of requirements. They should have good lubricating characteristics and low compressibility, a high aging resistance, and the influence of temperature on their viscosity should be as low as possible.

Hydraulic fluids are known that are hydraulic oils based on mineral oil. These fluids typically have a viscosity index of about 100. Additives are added to the mineral oil to ensure corrosion protection and to increase aging resistance. Viscosity index enhancers are frequently added to them as well. These are long-chain hydrocarbon compounds that have a low viscosity-increasing effect in more or less cold oils but dissolve in the oil at higher operating temperatures by uncoiling and thus increasing their volume. The oil thickens in the process, and the viscosity index increases as desired.

Such viscosity index enhancers have the disadvantage, however, that the long-chain hydrocarbon compounds are cracked into smaller fragments, which can sometimes change their original thickening effect dramatically. This effect is known as so-called permanent shearing loss among experts skilled in the art.

In addition, synthetic hydraulic fluids are known that may, for example, be composed of phosphate esters or anhydrous chlorinated hydrocarbons. Mixtures of both components are used as hydraulic fluids as well. Their viscosity index is approximately around 150.

Biodegradable hydraulic fluids based on vegetable oil have become known in the meantime. In particular, rapeseed oil (canola) is a known hydraulic fluid. The viscosity index of such vegetable oil-based hydraulic fluids is typically 200 and above. However, rapeseed oil is a poorly suited hydraulic fluid because of its unfavorable aging and hydrolytic properties.

The viscosity index is among the variables that are important for the efficiency of a hydraulic fluid. A higher viscosity index makes the hydraulic fluid thinner at low temperatures so that it can be pumped more easily, but it stays thicker at very high operating temperatures. Losses due to internal leakages are therefore reduced.

To increase the viscosity index synthetically, Evonik RohMax Additives GmbH, for example, has developed special polymer additives to be added to the hydraulic fluid and has introduced them into the market under the trade name “Dynavis”. Some tests have shown, however, that these polymers are sheared off after longer periods of useful life. As a result, the viscosity index drops during operation, which considerably reduces the desired increase in viscosity.

Based on the state of the art, the problem to be addressed by this invention is to provide a hydraulic fluid that comprises a constant high viscosity index greater than or equal to 200 even after a longer service life and in which the known issues of unfavorable aging and hydrolysis do not occur at all.

### SUMMARY

The problem of the prior art is solved by using vegetable oil with a natural viscosity index (VI) greater than or equal to 200, said oil having a portion of monounsaturated fatty acids of at least 80%, a portion of double unsaturated fatty acids of 1-10% at maximum, and a portion of triple unsaturated fatty acids of less than 1%, preferably less than 0.5% and particularly preferably less than or equal to 0.1%, as a pressure medium in hydraulic systems and/or as transmission fluid. As used herein, the term “natural viscosity index” means a viscosity index that results without adding any viscosity index enhancer.

According to the invention, the composition of the hydraulic fluid or transmission fluid has been optimized such that the portion of triple unsaturated fatty acids is kept extremely low at less than 1%, preferably less than 0.5%, and particularly preferably less than or equal to 0.1%. The outcome is an amazing and unprecedented stability of the hydraulic fluid according to the invention even for long service life.

The advantage of a constantly high viscosity index over a long service life has been described above. The hydraulic fluid or transmission fluid is thinner at lower temperatures and can be pumped more easily, while the fluid remains thicker at the very high operating temperatures in a pump. It should be mentioned that the energy balance shows a non-linear increase at a rising viscosity index. The effect is more pronounced for an increasing viscosity index, i.e., the difference in interval between 150 and 200 is greater than the one between 100 and 150.

Another advantage that the hydraulic fluid or transmission fluid according to the invention has shown over prior art mineral or synthetic hydraulic fluids is a significantly improved compressive modulus. It was found in tests that a piston in an hydraulic cylinder has to travel an approximately 10% shorter path to build the same pressure when the hydraulic oils according to the invention are used. This also results in shorter cycle times and less power demand, which has a high priority in industry in view of high energy costs and the decreasing profit margin calculations in the market. Lubrication of the hydraulic fluid or transmission fluid according to the invention is also improved as compared to the known mineral or synthetic hydraulic fluids. This reduces friction and influences power consumption favorably. Pump wear is also reduced.

Finally, pressure-viscosity behavior should be mentioned. The hydraulic fluid or transmission fluid according to the invention shows a significantly lower increase in viscosity under pressure than the known mineral or synthetic hydraulic fluids. This effect can be detected even in common hydraulic systems. Common hydraulic systems are systems that operate at pressures from 100 to 300 bar. This effect becomes even more apparent for the use as transmission fluid since the pressures involved here are much greater.

Another option is to use a portion of the vegetable oil in the form of an unsaturated ester of the vegetable oil. This is useful when the viscosity is to be changed based on the application requirements. When using pure vegetable oil, its viscosity is 40 Pas (pascal-seconds) at 40° C. Tests have shown that the viscosity can be reduced to 32 Pas at 40° C. when about 10% of the vegetable oil is replaced with the respective unsaturated ester. The unsaturated ester is used, as it were, to dilute the vegetable oil-based hydraulic fluid and therefore expands the range of uses in a simple manner.

The vegetable oil may optionally contain at least one additive selected from anti-oxidants, anti-corrosion agents, copper deactivators, wear protection agents and/or anti-foaming agents. The at least one additive is used to boost the already existing positive properties of the vegetable oil that may, e.g., in accordance with one embodiment of the invention, be due to a portion of an unsaturated ester and/or to at least minimize undesirable properties. The quantity of additive added depends on the application and can be from a few ppm (parts per million) to 2% or even up to 5%.

Anti-oxidants that provide aging protection through oxidation inhibition can be used as an additive. Both primary aging protecting agents in the form of radical interceptor NALs and secondary aging protecting agents as peroxide decomposers and passivators or metal ion deactivators can be used as anti-oxidants in the meaning of this invention. Other additives include anti-corrosion agents and rust protection additives. Surfactants that may be ashless or ash-building are particularly suitable as such additives.

Wear protection additives, also called EP/AW additives (extreme pressure/antiwear) should be mentioned here as well. These particularly include additives based on sulfur and phosphorus. While elemental sulfur was used initially, surface-active substances that contain zinc, phosphorus, and/or sulfur in their polar group are preferred nowadays. A well-known representative is zinc dithiophosphate (ZnDTP). ZnDTP also acts as an anti-aging and anti-corrosion agent. Other potential additives include copper deactivators and anti-foaming agents. Silicon oils are preferred as anti-foaming agents according to today's state of the art.

Various large-scale test studies have revealed that the use according to the invention of this or the vegetable oil(s) in one of the embodiments according to the invention resulted in excellent shear stability, measured over 20 hours, of 0.7% or less. Shear stability was −0.7% in some tests.

Other embodiments and advantages are described in the detailed description below. This summary does not purport to define the invention. The invention is defined by the claims.

### BRIEF DESCRIPTION OF THE DRAWINGS

The accompanying drawing illustrates embodiments of the invention.

FIG. 1 shows the energy consumption of the hydraulic fluid according to the invention compared to a known mineral oil when used as hydraulic fluid in an injection molding machine during one working cycle.

FIG. 2 shows the cycle times determined for the hydraulic fluid according to the invention compared to a known mineral oil when used as hydraulic fluid in an injection molding machine during one working cycle.

FIG. 3 is a table listing the viscosities of the novel hydraulic fluid versus those of the hydraulic oil of SAE class HLP 46 at various high pressures.

### DETAILED DESCRIPTION

Reference will now be made in detail to some embodiments of the invention, examples of which are illustrated in the accompanying drawings.

### I. Parent Product for Use as an Hydraulic Fluid

The base material used for a hydraulic fluid is a very-high-oleic-acid sunflower seed oil wherein the oil content, i.e., the content of monounsaturated fatty acids (oleic acid C18:1), was specially optimized and is 90.92%. The sunflower seed oil also comprises double unsaturated fatty acids (linoleic acid C18:2).

The content of triple unsaturated fatty acids (linolenic acid C18:3) in this embodiment was particularly reduced to trace amounts. Applicant performed studies that surprisingly demonstrated that the properties of the substance used as hydraulic fluid but also as transmission fluid particularly depend on the reduced amount of triple unsaturated fatty acids. Even very small constituent amounts of triple unsaturated fatty acids have an adverse effect on the overall behavior of the hydraulic fluid or transmission fluid. The content of triple unsaturated fatty acids was reduced to less than 0.1%. The hydraulic fluid according to the invention is readily and completely biodegradable.

A less optimal sunflower oil that is more commercially available can also be used as an hydraulic fluid. This alternative embodiment includes 83% monounsaturated oleic acid (C18:1), 8% double-unsaturated linoleic acid (C18:2), only a trace amount of triple-unsaturated linolenic acid (C18:3), 3.5% saturated palmitic acid (C18:0), 3.5% saturated stearic acid (C18:0), 1% saturated behenic acid (C22:0), and 0.5% saturated arachidic acid (C20:0). The aging characteristics of the hydraulic fluid depend on the proportions of the monounsaturated, double-unsaturated and triple-unsaturated fatty acids. The mono, double and triple unsaturated fatty acids degrade by comparative factors of 10:100:500, respectively. The aging process is a combination of oxidation, hydrolysis and polymerization of the fatty acids. Thus, a genetically modified sunflower oil having practically no linolenic acid (C18:3), over 90% oleic acid (C18:1) and the remainder consisting of linoleic acid (C18:2) is the best oil to used as an hydraulic fluid or transmission fluid. Although saturated fatty acids, such as palmitic acid and stearic acid impart good lubricating qualities to the hydraulic fluid, saturated fatty acids age faster than monounsaturated fatty acids and thus detract from the beneficial qualities of the hydraulic or transmission fluid.

Recently, sunflower oil has become available comprising more than 92% monounsaturated oleic acid, practically no triple-unsaturated linolenic acid, and the remainder nearly all double-unsaturated linoleic acid. Although it has long been recognized that certain plant oils have better lubricating qualities than mineral oils, these plant oils have been unsuitable for use as hydraulic fluids, transmission fluids and motor oils because the oils quickly degraded. Applicant recognized that it was the larger than trace amounts of triple-unsaturated linolenic acid and the unsaturated fatty acids present in the traditional plant oils that caused the oils to degrade. Applicant also recognized that new types of plant oils exist with low amounts of both triple-unsaturated linolenic acid and unsaturated fatty acids that are particularly suited for use as hydraulic fluids, transmission fluids and motor oils.

### II. Use as Hydraulic Fluid in an Injection Molding Machine

The hydraulic fluid with 90.92% monounsaturated fatty acids described under section I was used in the central hydraulic pump of a 2K SPM Arburg 520C injection molding machine. A hydraulic oil of SAE class HLP 46 was used for comparison. HLP 46 contains additives for increasing its aging resistance, its corrosion protection, and its EP properties. The measuring time was 81 cycles, 44 seconds per individual cycle. The material processed was ABS Schwarz Norodur 2K plastic. The nominal wattage of the hydraulic pump was 37,000.

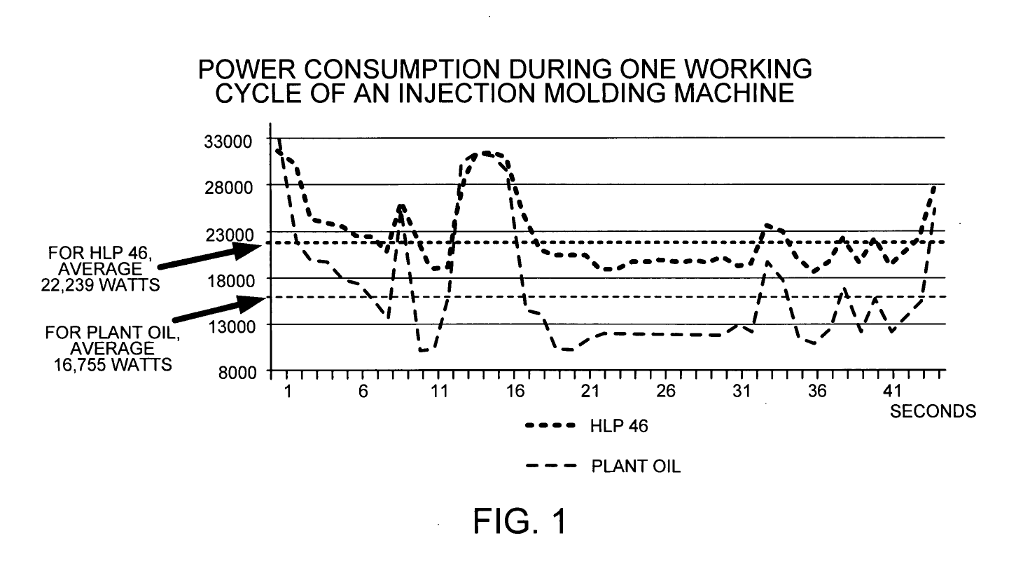

The power consumption of the injection molding machine was used as the criterion for comparing the performance of the two hydraulic fluids used. A power savings of 5,484 watts was achieved just by using the hydraulic fluid according to the invention versus HLP 46, which represents a power savings of about 25%. While the average power consumption when using HLP 46 totaled 22,239 watts, consumption measured for the hydraulic fluid of the invention was 16,755 watts. The different energy balance during one working cycle is shown in detail in FIG. 1.

### III. Use as Hydraulic Fluid in an Injection Molding Machine

The hydraulic fluid described under section I was used in the central hydraulic pump of a 2K SPM Arburg 420C injection molding machine. Once again, a hydraulic oil of SAE class HLP 46 was used for comparison, containing the additives listed under section II for increasing the aging resistance, the corrosion protection, and the EP properties of the oil

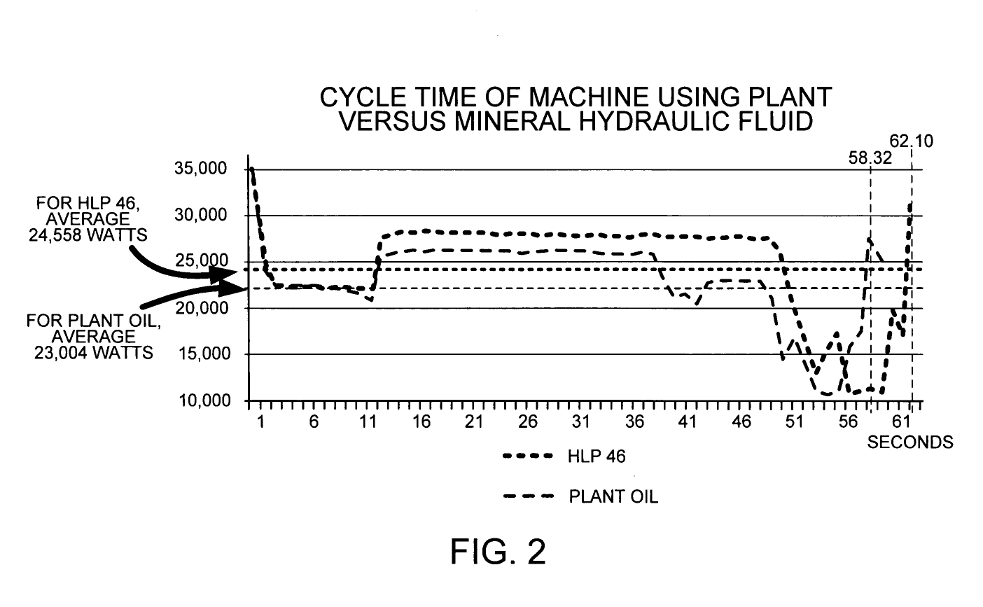

The cycle time of the injection molding machine was used as the criterion for comparing the performance of the two hydraulic fluids used. The measuring time was 59 cycles at 62.10 seconds per cycle for the hydraulic oil of SAE class HLP 46, compared to 58.32 seconds for the hydraulic fluid of the invention. This represents a 6.08% shortening of cycle time for the hydraulic fluid according to the invention. A thermoplastic elastomer was processed during the test. The nominal wattage of the hydraulic pump was 30,000. FIG. 2 illustrates the differences between the two hydraulic fluids used. In addition, energy savings of about 6.33% were achieved by using the hydraulic fluid according to the invention versus HLP 46. Also detected were significantly reduced CO₂ emissions of the injection molding machine.

### IV. Measuring the Wear Values of the Hydraulic Fluid of the Invention in a Reichert Scale

The hydraulic fluid according to the invention, as defined in section I was used without any additives and was tested in a Reichert scale. The wearing surface was only 12.61 mm² as compared to approximately 50 mm² for mineral oils.

### V. Pressure Stability Test of the Hydraulic Fluid According to the Invention Vs. Mineral Oils

The pressure stability test performed resulted in a value of about 10 (dimensionless) for the hydraulic fluid of the invention, while the value found for the mineral oil tested was about 7. It follows from the results of these tests, as specified in sections IV and V, that the hydraulic fluid of the invention as such, without any of the otherwise common additives, already meets all of the requirements of the accepted standard DIN 51 525 with respect to lubrication characteristics. In general, none of the otherwise common additives is needed. However, such additives can further improve these extremely positive findings, especially with respect to long-term stability. An optional additive is an anti-oxidant as an anti-aging agent.

### VI. Example for Hydraulic Applications with a Low Temperature Profile

The test was performed in an hydraulic servomotor for a servo-drive for controlling through-way and three-way valves in district heating systems. District heating systems need control valves that can handle extreme differential pressures. The drive is to ensure that high differential pressures can be handled at through-way valves, especially those with great nominal widths. The temperature profile was in the range of about 30° C. to 50° C. The hydraulic fluid of the invention was used without additives. The novel hydraulic fluid is particularly suited for this application due to its greater compressive modulus and better pressure-viscosity characteristic as compared to other lubricants. As a result, the control mechanism works more precisely, and the medium is thickened less under high pressure.

### VII. Sample Application in a High-Pressure Test Rig

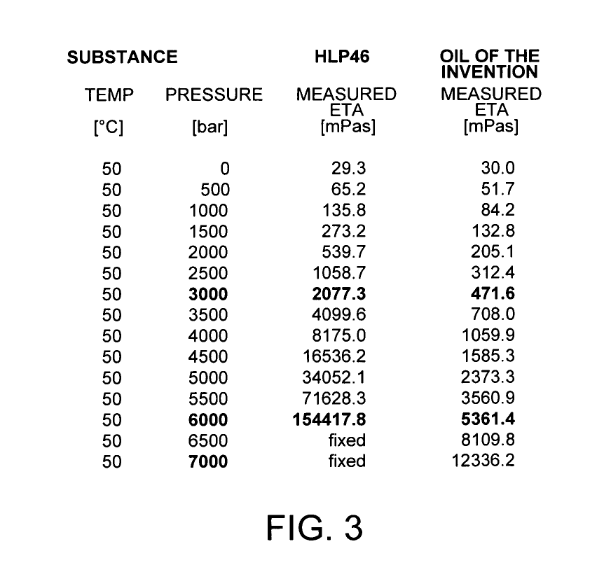

High-pressure test rigs are needed to test the proper functioning of injection nozzles in advance. Advanced injection nozzles work at pressures of up to 6,000 bar. The hydraulic apparatus for testing the nozzles is typically equipped with an hydraulic fluid. Mineral oil-based hydraulic oils would cause problems in these new test rigs because the mineral oils become highly viscous and even solid in limit ranges. Using a medium based on the hydraulic fluid according to the invention provides an ideal solution to the problem because the hydraulic fluid according to the invention is sufficiently aging-resistant and ensures regular lubrication as it remains more liquid. FIG. 3 shows an overview of the viscosities of the hydraulic fluid according to the invention versus those of the hydraulic oil of SAE class HLP 46 determined in the test. FIG. 3 shows that the viscosity of the mineral oil HLP 46 is more than 2 Pascal at 3000 bar, while the viscosity of the vegetable oil of section I remains below 500 milli-Pascal at 3000 bar. In addition, the viscosity of the mineral oil HLP 46 is more than 154 Pascal at 6000 bar, while the viscosity of the vegetable oil of section I remains below 6 Pascal at 6000 bar.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

What is claimed is:

1. A method comprising:
using a vegetable oil as a pressure medium in an hydraulic system, wherein the vegetable oil has a natural viscosity index greater than or equal to 200, wherein the vegetable oil has a first portion of monounsaturated fatty acids of at least 80% by weight, a second portion of double unsaturated fatty acids of no more than 10% by weight, and a third portion of triple unsaturated fatty acids of less than 1% by weight.

2. The method of claim 1, wherein a portion of the vegetable oil is used in the form of an unsaturated ester of the vegetable oil.

3. The method of claim 1, wherein the vegetable oil contains an additive taken from the group consisting of: an anti-oxidant, a copper deactivator, an anti-corrosion agent, a wear-protection agent, and an anti-foaming agent.

4. The method of claim 3, wherein the maximum amount of any one additive in the vegetable oil is less than 5% by weight.

5. The method of claim 1, wherein the vegetable oil exhibits a shear stability, and wherein the shear stability measured over 20 hours of use is 0.7% or less.

6. The method of claim 1, wherein the hydraulic system operates at a pressure of 100 to 300 bar.

7. A method comprising:
using a vegetable oil as a transmission fluid, wherein the vegetable oil has a natural viscosity index greater than or equal to 200, wherein the vegetable oil has a first portion of monounsaturated fatty acids of at least 80% by weight, a second portion of double unsaturated fatty acids of no more than 10% by weight, and a third portion of triple unsaturated fatty acids of less than 1% by weight.

8. The method of claim 7, wherein a portion of the vegetable oil is used in the form of an unsaturated ester of the vegetable oil.

9. The method of claim 7, wherein the vegetable oil contains an additive taken from the group consisting of: an anti-oxidant, a copper deactivator, an anti-corrosion agent, a wear-protection agent, and an anti-foaming agent.

10. The method of claim 9, wherein the maximum amount of any one additive in the vegetable oil is less than 5% by weight.

11. The method of claim 7, wherein the vegetable oil exhibits a shear stability, and wherein the shear stability measured over 20 hours of use is 0.7% or less.

12. A method comprising:
using sunflower oil in an hydraulic system, wherein the sunflower oil has less than 0.1% linolenic acid by weight, over 90% oleic acid by weight and the remainder comprising mostly linoleic acid by weight.

13. The method of claim 12, wherein the sunflower oil has a natural viscosity index greater than or equal to 200.

14. The method of claim 12, wherein less than 1% by weight of the sunflower oil is comprised of saturated fatty acids.

15. The method of claim 12, wherein the sunflower oil exhibits a shear stability, and wherein the shear stability measured over 20 hours of use is 0.7% or less.
